(19) United States
(12) Patent Application Publication
(10) Pub. No.: US 2008/0249074 A1
(43) Pub. Date: Oct. 9, 2008

(54) Composition for the Transdermal Administration of Physiologically Active Agents

(75) Inventors: Luis Carvajal Martin, Madrid (ES); Francisco Sevilla Tirado, Zaragoza (ES); Yolanda Aznar Antonanzas, Zaragoza (ES)

Correspondence Address: BERENBAUM, WEINSHIENK & EASON, P.C, 370 17TH STREET, SUITE 4800, DENVER, CO, 80202, US

(73) Assignee: LABORATORIOS BELMAC, S.A., San Sebastian de los Reyes (ES)

(21) Appl. No.: 12/092,551

(22) PCT Filed: Nov. 3, 2006
(86) PCT No.: PCT/EP06/68054

§ 371 Date: May 2, 2008

(30) Foreign Application Priority Data
Nov. 3, 2005 (ES) P 200502711

(51) Int. Cl. A61K 47/44; A61K 47/12; A61K 31/351; A61K 31/07; A61K 31/59; A61K 31/355; A61K 47/22

(52) U.S. Cl. 514/167; 514/784; 514/772; 514/460; 514/725; 514/458

(57) ABSTRACT

Improved composition for the administration of a physiologically active agent through the skin which comprises at least one vegetable oil, oleic acid, and at least one lactone of a saturated or unsaturated fatty acid of 5 to 22 carbon atoms, said two last components acting as absorption enhancers. Using said composition, unexpected increased values of the absorption of the physiologic active agent through the skin are obtained due to an unpredictable synergistic effect.

The present invention relates to a composition for the transdermal administration of a physiologically active agent.

### BACKGROUND ART

The administration of physiologically active agents through the skin provides some advantages over conventional means of oral and systemic administration, avoiding disadvantages such as variable absorption indexes, the metabolic degradation of the drug and gastrointestinal irritation, among others. Moreover, transdermal administration allows the use of physiologically active agents with a short half-life, at reduced doses, at high local concentrations and a controlled release of the physiologically active agent for a long period of time.

Skin is a relatively thick and structurally complex membrane which acts as a barrier avoiding both the absorption of water and electrolytes and their loose. The cells of the stratum corneum are the main barrier against the absorption of physiologically active agents which have been administered through the skin. The stratum corneum is a thin, although dense layer of hydrophilic highly keratinized cells. The intercellular spaces are full of strongly hydrophobic lamellar lipids. The combination of the hydrophilic keratinized cells and the hydrophobic intercellular material constitute a barrier against both hydrophilic and hydrophobic substances. The skin pH varies between 4 and 6, due to the sweat and the evaporation thereof, but, exceptionally, pH could reach alkaline values due to pathological states.

On the other hand, not all of the physiologically active agents diffuse in a similar way through the skin barrier. Said diffusion depends on the physical and chemical features of the molecule.

For an absorption enhancer to be effective, the physiologically active agent has to be dissolved in said enhancer or in the phase where the enhancer is soluble. When the physiologically active agent is in a “non-ionic” form and it is dissolved in an oily phase, the absorption is relatively high. If the physiologically active agent is in an ionized form and dissolved in an aqueous phase, the absorption would be determined by the oil:water partition coefficient of the physiologically active agent. On the other hand, when water is the solvent and the composition is spread over the skin, water evaporates and the physiologically active agent precipitates, becoming incapable to pass through the epithelial barrier.

Among the proposed options to overcome said problem, there is the use of polyol type wetting agents (e.g. glycerine, prophyleneglycol, diethylene glycol monoethyl ether) which delays the evaporation of water and improve the oil-aqueous partition coefficient. Polyols have the disadvantage that they are hardly absorbed through the skin and that provide a greasy sensation on the skin.

Another option to ease the pass of an active ingredient through the skin is to add one or more absorption enhancers to the topical compositions for transdermal administration. A large number of per-cutaneous absorption enhancers have been described in the literature, which, ideally, are innocuous compounds that ease the diffusion of the active ingredient through the stratum corneum of the skin (cf. Barry B W et al. J. Pharm. Pharmacol. 1987, vol. 39(7), pp. 535-46). Thus, for example, it is known that oleic acid may act as an absorption enhancer (ibid). European Patent EP 248.885 discloses compositions comprising a group of cyclic esters and lactones as absorption enhancers through the skin and through several body membranes. Among other compounds, the use of 15-pentadecanolactone as an absorption enhancer is disclosed. Patent application WO 03/28702 discloses topical formulations in the form of emulsions and creams containing a salt of ibuprofen as emulsifier and 15-pentadecanolactone as an absorption enhancer. Thus, since ibuprofen is present as a salt, it will be dissolved in the aqueous phase.

Nevertheless, some physiologically active agents in the form of creams, ointments and unguents have still difficulties to pass through the epidermis to reach the dermis and to be distributed through the bloodstream. Thus, it is very difficult to formulate an oil which is capable of wetting the skin and being absorbed through it without using emulsifiers.

Thus, from the state of the art arises that there is still a need of find new compositions for transdermal administration which allow the effective liberation through the skin of a physiologically active agent.

### SUMMARY OF THE INVENTION

Although it is already known that oleic acid and some lactones may act as an absorption enhancers in compositions for transdermal administration, the use of their combination has never been suggested. The inventors have surprisingly found that when adding oleic acid and at least one lactone of a saturated or unsaturated fatty acid comprising 5 to 22 carbon atoms to a composition for transdermal administration comprising a physiologically active agent in a suitable vehicle, the values of the absorption of the physiologically active agent through the skin are higher than expected due to a unexpected synergistic effect.

Thus, one aspect of the invention is related to a composition for the transdermal administration of a physiologically active agent comprising an effective amount of at least one physiologically active agent, at least one vegetal oil, oleic acid and at least one lactone of a saturated or unsaturated fatty acid comprising from 5 to 22 carbon atoms.

As a consequence of the presence of oleic acid together with at least one of said lactones as an absorption enhancer, the composition of the present invention shows improved skin wetting power and absorption capacity through the skin. Moreover, they are advantageous because they can be applied directly without the need of an occlusive dressing to obtain a quick absorption. Another advantage is that said mixture of absorption enhancers allows to reduce the cost of the composition because the price of oleic acid is much lower than the price of said lactones. In addition, the persistent balsamic odour, which can be refused by a large percentage of people, decreases when lowering the proportion of said lactones.

Also forms part of the invention a method to promote the penetration of physiologically active agents through the skin of a human or animal body. Said method comprises the administration over the skin of a human or an animal of a composition for transdermal administration according to the present invention.

The term “physiologically active agent” relates to a chemical compound which induces a physiological or pharmacological effect and includes therapeutically effective agents, prophylactic effective agents or cosmetically effective agents. The term “composition for transdermal administration” relates to a composition which is absorbed through the skin with the purpose of supply a physiologically active agent to a place or tissue which is adjacent or distant from the application place. The term “vegetal oil” relates to each of the oils obtained from seeds and fruits of vegetables, formed by glycerine esters and fatty acids.

### DETAILED DESCRIPTION OF THE INVENTION

In a preferred embodiment of the composition for transdermal administration of the present invention the vegetal oil, which acts as a fatty solvent, is present in an amount of between about 10% w/w and about 80% w/w, the oleic acid is present in an amount of between about 2% w/w and about 50% w/w, and said lactone or mixture of lactones is present in an amount of between about 2% w/w and about 10% w/w. Preferably, the ratio of lactone or mixture of lactones with regard to oleic acid is from 0.8:1 to 1:10.

In another preferred embodiment of the composition for transdermal administration of the present invention, the lactone used as an absorption enhancer comprises from 12 to 18 carbon atoms. The composition can comprise one or more of said lactones. In a more preferred embodiment, the lactone is 15-pentadecanolactone (CPE-215).

In another preferred embodiment, the vegetal oil is selected from a vegetal oil with a content of oleic acid higher than 60% w/w, and a vegetal oil with a content of linolenic acid or another 3-omega fatty acid of at least about 10% w/w. Preferably, the vegetal oil contains a low content of linoleic acid and of palmitic acid. More preferably, the vegetal oil is selected from olive oil and primrose oil.

Further, the mixture of said lactones and oleic acid improves the solubility of some physiologically active agents in oily mediums, such as oleuropein, acidic non-steroid anti-inflammatory drugs, polyphenols and liposoluble vitamins. Green tea derivates, oak galls derivatives, lutein and derivatives thereof, quercetin and derivatives thereof, and naringenin and derivates thereof, are included among the polyphenols. Preferably, vitamin A, vitamin D, vitamin E and vitamin K are included among the liposoluble vitamins. Preferably, indometacin, sulindac, mefenamic acid and derivatives thereof, tolmetin, ketorolac, diclofenac, ibuprofen, naproxen and ketoprofen are included among the non-steroid anti-inflammatory drugs.

In another preferred embodiment, the composition for transdermal administration of the present invention comprises oleuropein as physiologically active agent. Preferably the amount of oleuropein is from about 0.1% w/w to about 0.4% w/w with regard to the total of the ingredients of the composition. The compositions for transdermal administration of oleuropein of the present invention avoid the disadvantages related to the hardening and keratinisation effects on the skin that other topical compositions cause.

In some cases, the solubilisation of the physiologically active agent will demand for the use of a non-volatile co-solvent. Diethylene glycol monoethyl ether (Transcutol®), a polyol comprising from 2 to 15 carbon atoms and from 2 to 10 hydroxyl groups, such as propylene glycol, or other suitable co-solvent capable of getting dissolved in the composition of the present invention are among the non-volatile co-solvents that can be used for the improvement of the solubilisation of some of the active agents present in the composition of the present invention. Usually, the co-solvent is employed in a proportion up to 20% w/w with regard to the mixture of vegetal oil and absorption enhancers. Preferably, the co-solvent is diethylene glycol monoethyl ether or propylene glycol. Preferably, the amount of co-solvent employed is the minimum required to solubilise the physiologically active agent without altering the absorption rate.

Depending on the properties of the physiologically active agent, the solubilisation in the composition can be achieved by one of the following methods:

Method A. The physiologically active agent is dissolved in a suitable solvent (ethanol, acetone or other volatile solvent); then, the resulting solution is dispersed into a mixture of at least one vegetal oil and the absorption enhancers, i.e. at least one lactone of a saturated or unsaturated fatty acid comprising from 5 to 22 carbon atoms and the oleic acid, and, subsequently, the solvent is removed from the mixture under reduced pressure.

Method B. The physiologically active agent is dissolved in a non-volatile co-solvent which, then, is added to the mixture of vegetal oil and absorption enhancers under continuous stirring.

The composition for transdermal administration further comprises suitable amounts of pharmaceutically or physiologically acceptable excipients. Thus, the composition can comprise preservatives, melting-point regulators, emollients, emulsifiers, flavours, and mixtures thereof. Suitable amounts of said additional components can be added to the composition of the present invention in order to improve the specific properties thereof, as will became apparent to those skilled in the art.

Methylparaben, ethylparaben, propylparaben, butylparaben and benzoic acid are included among the preservative agents which can be used in the composition for transdermal administration of the present invention.

Cetyl alcohol, stearic alcohol, cetylstearyl alcohol and C₁₄-C₁₈ fatty acid monoesters of glycerine are included among the possible melting-point regulators.

Among the suitable emulsifiers, sorbitan monoesters of fatty acids, i.e. sorbitan monostearate, sorbitan monolaurate, sorbitan monopalmitate and sorbitan monooleate, and polyoxyethylene sorbitan monoesters of fatty acids, i.e. polyoxyethylene sorbitan monooleate (Polysorbate 20), polyoxyethylene sorbitan monopalmitate (Polysorbate 40), polyoxyethylene sorbitan monostearate (Polysorbate 60) and polyoxyethylene sorbitan monooleate (Polysorbate 80) are included.

The composition for transdermal administration of the present invention shows a weak acid character. Said weak character acid confers to the composition a weak ionic capacity and a higher wetting and penetration capacity when getting in contact with the skin. Normally, the dispersion of 1 g of the composition into 100 ml of water provides a pH between about 3 and about 5.5, close to the pH of the skin. Preferably, the composition for transdermal administration shows a pH between about 5 and 5.5.

The composition for transdermal administration of the present invention can be formulated in the form of an oil, an unguent, an ointment, a cream, an emulsion or a paste.

In the case of an oil, the composition for transdermal administration of the present invention comprises from about 20% w/w to about 80% w/w of vegetal oil, preferably virgin olive oil. A more preferred oil composition according to the present invention further comprises from about 2.0% w/w to about 20.0% w/w of a co-solvent. Preferably, the composition additionally comprises from about 0.05% w/w to about 0.2% w/w of an emulsifier and, optionally, up to 0.1% w/w of a preservative agent.

A particularly preferred composition in the form of oil according to the present invention comprises:
(i) a physiologically effective amount of a physiologically active agent;
(ii) from about 2.0% w/w and about 10.0 w/w of 15-pentadecanolactone;
(iii) from about 2.0% w/w and about 50.0 w/w, preferably from about 2.0% w/w and about 10.0 w/w, of oleic acid;
(iv) from about 5.0% w/w and about 20.0 w/w of co-solvent;
(v) from about 0.05% w/w and about 0.2% w/w of emulsifier;
(vi) optionally, up to about 0.1% w/w of a preservative agent; and
(vii) virgin olive oil in an enough amount to equal 100% w/w of the composition.

In the case of an unguent, the composition for transdermal administration of the present invention comprises from about 10% w/w and about 70% w/w of vegetal oil, preferably virgin olive oil, and from about 5.0% w/w and about 25.0% w/w of a melting-point regulator. Preferably, the composition comprises from about 5.0% w/w and about 50.0% w/w, more preferably from about 5.0% w/w and about 10.0% w/w of acid oleic.

A more preferred composition in the form of an unguent according to the present invention further comprises from about 2.0% w/w and about 20.0% w/w of co-solvent, and from about 0.1% w/w and about 3.0% w/w of an emulsifier.

A particularly preferred composition in the form of an unguent according to the present invention comprises
(i) a physiologically effective amount of a physiologically active agent;
(ii) from about 2.0% w/w and about 10.0 w/w of 15-pentadecanolactone;
(iii) from about 5.0% w/w and about 50.0 w/w, preferably between about 5.0% w/w and about 10.0 w/w, of oleic acid;
(iv) from about 2.0% w/w and about 20.0 w/w of co-solvent;
(v) from about 5.0% w/w and about 25.0% w/w of a melting-point regulator;
(vi) from about 0.1% w/w and about 3.0% w/w of an emulsifier;
(vii) virgin olive oil in an enough amount to equal 100% w/w of the composition.

In the case of a cream, the composition for transdermal administration of the present invention comprises from about 10% w/w and about 50% w/w of vegetal oil, preferably virgin olive oil, and from about 1.0% w/w and about 3.0% w/w of at least one emulsifier, and water in an enough amount to equal 100% w/w of the composition, typically between about 40% w/w and about 60% w/w.

A more preferred composition in the form of a cream according to the present invention further comprises from about 5% w/w and about 25% w/w of a melting-point regulator.

A more preferred composition in the form of a cream according to the present invention comprises:
(i) a physiologically effective amount of a physiologically active agent;
(ii) from about 2.0% w/w and about 10.0 w/w of 15-pentadecanolactone;
(iii) from about 5.0% w/w and about 50.0 w/w, preferably between about 5.0% w/w and about 10.0 w/w, of oleic acid;
(iv) from about 2.0% w/w and about 20.0 w/w of co-solvent;
(v) from about 10.0% w/w and about 50.0% w/w, preferably from about 10.0% and about 20.0 %, of virgin olive oil;
(vi) from about 5.0% w/w and about 25.0% w/w of at least one melting-point regulator;
(vii) from about 1.0% w/w and about 3.0% w/w of at least one emulsifier;
(viii) from about 0.15% w/w and about 0.4% w/w of at least one conservative agent, and
(ix) water in an enough amount to equal 100% w/w of the composition.

The composition of the present invention allows the effective absorption of the physiologically active agent, and addicitonally, a high proportion of the physiologically active agent is absorbed through the skin. The effective amount of physiologically active agent to provide the desired effect will be determined by a person skilled in the art, according to usual practice.

After the absorption through the skin of the composition of the present invention, the active ingredient will be released depending on several factors, such as the hydrophilic/lipophilic balance (HLB) of the cream, the diameter of the fatty globules of the cream, and the partition coefficient of the active ingredient in its non-dissociated state, among others. According to these parameters, the adjustement of the proportions of the ingredients of the composition will allow to optimize the release of the active ingredient from the fatty matrix thereof depending on the physical and chemical features of said active agent.

Throughout the description and claims the word “comprise”, and variations thereof, is not intended to exclude other technical features, additives, components, or steps. Unless otherwise stated, all the percentages and proportions are given on a weight basis. The percentages are by weight of the total composition.

Additional objects, advantages and features of the invention will become apparent to those skilled in the art upon examination of the description or may be learned by the practice of the invention.

### BRIEF DESCRIPTION OF THE DRAWINGS

FIG. 1 shows the effect of the topical administration of ibuprofen by the application of the compositions designated as Vehicle I, II, III and IV, in comparison with the topical administration of water, corresponding to the blank-vehicle (B), over the development of the plantar edema induced by the injection of carrageenan in rats. Ordinate axis represents the % of increment of volume versus the initial volume (V₀ corresponding to the time just before the injection of carrageenan) and the abscissa axis represents the time when the measure was performed, i.e. 60 minutes after the injection of carrageenan (V₁).

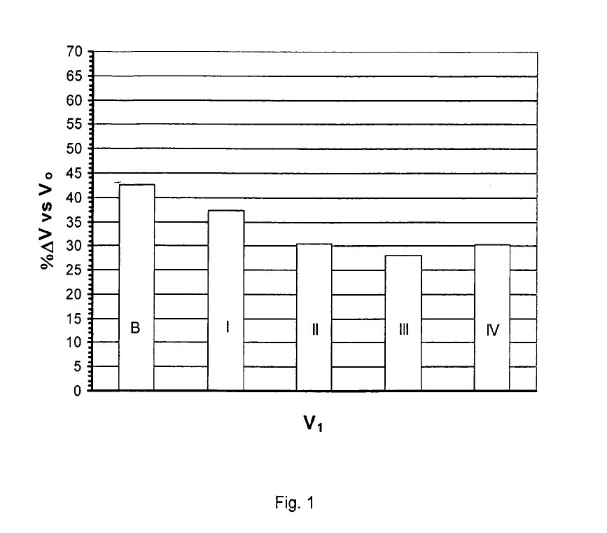

The following examples and drawings are provided by way of illustration, and are not intended to be limiting of the present invention.

### DETAILED DESCRIPTION OF PARTICULAR EMBODIMENTS

### EXAMPLE 1

### Preparation of Composition in the Form of an Oil

25 kg of a composition were prepared by the following process:

a) Preparation of the Aqueous Phase.

62.5 g of 40% oleuropein w/w (a 0.1% w/w of oleuropein), 5,000 g of diethylene glycol monoethyl ether, and 12.5 g of benzoic acid were added to a stainless steel tank. The mixture was stirred until complete dissolution.

b) Preparation of the Oily Phase.

The remaining components, i.e. 17,120 g of virgin olive oil, 1,250 g of oleic acid, 1,500 g of 15-pentadecalactone, and 25 g of sorbitan monoestarate (Span 60), were added to a second stainless steel tank. The mixture was heated between 60 and 65° C. under continuous stirring for about 30 minutes more, until total homogenization.

c) Obtaining the Oil

The mixture obtained at step b) was added over the mixture obtained at step a) under stirring. 22.5 g of bergamot oil, and 7.5 g of tea-tree aroma were added as flavours and the mixture was homogenized under stirring until it cooled down to room temperature.

Following the same process, three additional oil compositions containing a 0.2% w/w, a 0.4% w/w and a 0.8% w/w of oleuropein, respectively, were prepared.

### EXAMPLE 2

### Preparation of a Composition in the Form of an Oil

50 g of ibuprofen were dissolved in 200 g of ethanol (the amount needed to dissolve the active ingredient; ethanol is not an ingredient of the composition) under stirring. Next, said mixture was added under continuous stirring to a mixture of 150 g of CPE-215, 75 g of oleic acid, and 2,225 g of virgin olive oil. The mixture was heated between about 35-40° C. and the ethanol was removed under reduced pressure up to a content below to 0.5% w/w.

### EXAMPLE 3

### Preparation of a Composition in the Form of an Oil

50 g of ibuprofen were dissolved in 250 g of diethylene glycol monoethyl ether. Next, said mixture was added under continuous stirring to a mixture of 150 g of CPE-215, 125 g of oleic acid, and 1,925 g of virgin olive oil.

### EXAMPLE 4

### Preparation of a Composition in the Form of an Unguent

25 kg of a composition were prepared by the following process:

a) Preparation of the Aqueous Phase.

250.0 g of 40% oleuropein w/w (a 0.4% w/w of oleuropein), and 500 g of diethylene glycol monoethyl ether were added to a stainless stain tank. The mixture was stirred until total dissolution.

b) Preparation of the Oily Phase

14,470 g of virgin olive oil, 2,500 g of oleic acid, 750 g of 15-pentadecalactone, 6,000 g of cetyl alcohol and 500 g of sorbitan monoestarate (Span 60) were added to a second stainless steel tank. The mixture was heated until approximately 50° C. under continuous stirring until total homogenization.

c) Obtaining the Unguent

The mixture obtained in step b) was added over the mixture obtained in step a) under stirring. 22.5 g of bergamot oil and 7.5 g of tea-tree aroma were added as flavours and the mixture was homogenized under stirring until it cooled down to room temperature.

Following the previous process, two additional oil compositions containing 0.1% w/w and 0.2% w/w of oleuropein, respectively, were prepared.

### EXAMPLE 5

### Preparation of a Composition in the Form of a Cream

25 kg of a composition were prepared by the following process:

a) Preparation of the Aqueous Phase.

25 g of metilparaben, 12.5 g of propilparaben, 240 g of polyoxiethylene sorbitan monoestearate (Tween-60) and 12.6 L of water were added to a stainless stain tank. The mixture was stirred at about 50° C. until total dissolution.

b) Preparation of the Oily Phase

6,250 g of virgin olive oil, 1,250 g of oleic acid, 1,500 g of 15-pentadecalactone, 1,875 g of cetyl alcohol, and 260 g of sorbitan monoestarate (Span 60), were added to a second stainless steel tank. The mixture was heated at about 50° C. under continuous stirring until total homogenization.

c) Dissolution of the Physiologically Active Agent

125.0 g of 40% oleuropein w/w (a 0.2% w/w of oleuropein) were dispersed in 595 g of diethylene glycol monoethyl ether at room temperature.

d) Obtaining the Cream

The mixture obtained in step b) was added over the mixture obtained in step a) under stirring in an Ultra-Turrax type system until the homogenization of the mixture. Next, the mixture obtained in step c) was added under stirring together with 12,3 g of Shine aroma and it was let to cool down until room temperature.

Following the previous process, two additional cream compositions containing 0.1% w/w and 0.4% w/w of oleuropein, respectively, were prepared.

### EXAMPLE 6

### Preparation of a Composition in the Form of a Cream

25 kg of a composition were prepared by the following process:

a) Preparation of the Active Phase:

150 g of sodium diclofenac were dissolved in 17.5 L of water. 6,250 g of virgin olive oil and 1,250 g of oleic acid were added to another tank. Both tanks were heated until approximately 50° C. and, then, the oily mixture was added to the solution of diclofenac and the pH was adjusted between about 5 and about 5.5 with 0.1 N hydrochloric acid.

Once the mixture was homogenized, it was left to stand and the phases were separated. The aqueous phase was discarded and 750 g of Transcutol was added while maintaining the solution at a temperature of about 50° C.

b) Obtaining the Cream

1,500 g of 15-pentadecanolactone, 1,875 g of cetyl alcohol and 260 g of sorbitan monoestearate (Span 60) were added to the oily mixture. The mixture was heated to about 50° C. under continuous stirring in a Ultra-Turrax system for about 30 minutes. Then, 12.3 g of Shine aroma were added and the mixture was let to cool down until room temperature.

### EXAMPLE 7

### Stability of the Compositions in the Form of an Oil with 0.1% 0.2% and 0.4% w/w of Oleuropein

The stability through the time of the three oils obtained in Example 1 was evaluated by testing the compositions under different conditions of temperature and humidity. Different samples of each one of the 0.1%, 0.2% and 0.4% w/w oleuropein compositions were stored in climate chambers at about 25° C. (real-time test of stability), at about 30° C. (medium stability test), and at about 40° C. (accelerated stability test), at a relative humidity of about 60%, about 65%, and about 75%, respectively. The samples tested in the accelerated stability test were stable up to a period of 6 months. The rest of the samples did not show a significant variation for the first 12 months.

### EXAMPLE 8

### Study of the Absorption through the Skin of a Composition for Transdermal Administration of the Present Invention containing Ibuprofen

The objective of this study is to determinate the absorption through the skin of a physiologically active agent by the use of the composition for transdermal administration of the present invention. So, the anti-inflammatory effect of the substance to be tested was evaluated when the composition of the present invention was applied transdermally.

8.1. Method for the Determination of the Absorption through the Skin

For the study of the transdermal absorption, a test was performed to evaluate the anti-inflammatory activity of ibuprofen with different absorption enhancers in the same base-vehicle over the carrageenan-induced rat paw plantar edema. Said anti-inflammatory effect was used as an indicator of the absorption of the active ingredient through the skin.

50 male Wistar rats, with a weight of about 175 g, were used in the test. The animals were acclimatized to the place where the tests were carried out for at least 4 days under a controlled temperature of 22° C. and a relative humidity between 50 and 75%, with an exchange of filtered fresh air of about 10 times per hour, and with light cycles of 12 h. The animals were fed ad libitum with and standard diet and drinking water as beverage. The test comprised 5 test groups of 10 animals each. Blank-vehicle (water) and the compositions designated as Vehicle I, Vehicle II, Vehicle III and Vehicle IV, which compositions are shown in Table 1, were applied to the animals.

TABLE 1
Test Compositions

| | Vehicle I | | Vehicle II | | Vehicle III | | Vehicle IV | |
|---|---|---|---|---|---|---|---|---|
| Ibuprofen | 5.0 | g | 5.0 | g | 5.0 | g | 5.0 | g |
| CPE-215 | 15.0 | g | — | | 15.0 | g | — | |
| Transcutol | 100.0 | g | 100.0 | g | 100.0 | g | 100.0 | g |
| Oleic acid | — | | 25.0 | g | 25.0 | g | — | |
| Benzoic acid | 0.3 | g | 0.3 | g | 0.3 | g | 0.3 | g |
| Span-60 | 0.5 | g | 0.5 | g | 0.5 | g | 0.5 | g |
| Virgin Olive Oil | 379.3 | g | 369.3 | g | 354.3 | g | 394.3 | g |
| Bergamot Oil | 5.0 | mL | 5.0 | mL | 5.0 | mL | 5.0 | mL |
| Tea-tree Oil | 1.8 | mL | 1.8 | mL | 1.8 | mL | 1.8 | mL |

The test was performed according to the method disclosed by Winter et al. (Proc. Soc. Exp. Biol. Med. 1962; vol. 111, pp. 544-547), adapted to the rat paw. The method consisted in the injection of 100 μl of a 0.75% carrageenan solution in physiological saline in the plantar face of the left hind paw of the rat. The volume of the paws was determined by a plethysmometer just before the injection of the carrageenan (V₀), 60 minutes after the injection (V₁) and 180 minutes (V₂) after the injection.

The tested substances were administered topically according to a modification of the method disclosed by Mass6 et al. (Rev. Farmacol. Clin. Exp. 1990, vol. 7, p. 85), following a blind and randomized trial. 100 μl of the substance to be tested were applied over the paw of the animal, spread over the whole surface of the paw, massaging until the complete absorption. The application was repeated 2 times, namely 18 h before the injection of carrageenan and 60 minutes before said injection. Simultaneously to the application, 30 ml/kg of distilled water were administered orally.

Once the results were obtained, the mean±standard error of mean (S.E.M.) of the volumes of the paws of the animals were calculated for the three observation periods. The average values of each experimental group were used for the determination of the average increase in percentage of the volume of the paws, as an effect of the injection of carrageenan during the periods of observation V₁ and V₂, with regard to the initial volume (V₀), and, subsequently, the percentages of the variation of said values with regard to the blank-vehicle group were determined. The evaluation of the statistic signification of the differences was performed by the Student's t-test for independent data, using a p<0.05 level to establish the difference criteria.

8.2. Results

The effect of the different vehicles over the anti-inflammatory activity of ibuprofen is shown in Tables 2 to 6. The results are shown as ml of volume of the paw.

For the evaluation of the transdermal absorption the values of the volume of the paws obtained 60 minutes after the injection of carrageenan (V₁) were considered, because the absorption after 180 minutes after the injection (V₂) could be influenced, on one hand, by the loose of the barrier effect of a so inflamed skin and, on the other hand, by the release of oleic acid (one of the tested absorption enhancers) from the olive oil, by the action of the esterases of the skin.

TABLE 2
Effect of the Blank-vehicle (water)

| Animal N^{o} | V₀ | V₁ | V₂ |
|---|---|---|---|
| 1 | 0.84 | 1.45 | 2.61 |
| 2 | 0.87 | 1.54 | 3.19 |
| 3 | 1.01 | 1.59 | 2.00 |
| 4 | 0.87 | 1.33 | 2.17 |
| 5 | 1.16 | 2.17 | 3.33 |
| 6 | 1.01 | 2.43 | 3.19 |
| 7 | 0.93 | 1.30 | 3.33 |
| 8 | 1.01 | 2.09 | 2.90 |
| 9 | 0.87 | 1.45 | 3.10 |
| 10 | 1.16 | 1.59 | 3.48 |
| Mean ± S.E.M. | 0.97 ± 0.04 | 1.70 ± 0.12 | 2.93 ± 0.16 |

| | | |
|---|---|---|
| increase % vs V₀ | 42.56 | 66.77 |

TABLE 3
Effect of Vehicle I

| Animal N^{o} | V₀ | V₁ | V₂ |
|---|---|---|---|
| 1 | 0.87 | 1.30 | 1.30 |
| 2 | 0.99 | 1.71 | 1.77 |
| 3 | 1.25 | 1.80 | 2.29 |
| 4 | 1.25 | 1.88 | 2.14 |
| 5 | 1.07 | 2.17 | 2.03 |
| 6 | 1.01 | 2.03 | 1.68 |
| 7 | 1.01 | 1.25 | 1.62 |
| 8 | 1.04 | 1.74 | 1.54 |
| 9 | 1.01 | 1.45 | 1.45 |
| 10 | 0.87 | 1.22 | 1.45 |
| Mean ± S.E.M. | 1.04 ± 0.04 | 1.66 ± 0.11 | 1.73 ± 0.10 |

| | | |
|---|---|---|
| increase % vs. V₀ | 37.30 | 39.93 |
| inhibition % vs. blank-vehicle | 12.36 | 40.19 |

TABLE 4
Effect of Vehicle II

| Animal N^{o} | V₀ | V₁ | V₂ |
|---|---|---|---|
| 1 | 1.04 | 1.57 | 1.91 |
| 2 | 1.07 | 1.57 | 1.65 |
| 3 | 0.93 | 1.33 | 1.71 |
| 4 | 1.33 | 1.42 | 2.17 |
| 5 | 1.16 | 1.80 | 1.88 |
| 6 | 1.16 | 1.94 | 1.51 |
| 7 | 1.13 | 1.71 | 1.88 |
| 8 | 1.01 | 1.62 | 1.80 |
| 9 | 1.04 | 1.30 | 1.74 |
| 10 | 0.90 | 1.28 | 1.28 |
| Mean ± S.E.M. | 1.08 ± 0.04 | 1.55 ± 0.07 | 1.75 ± 0.08 |

| | | |
|---|---|---|
| increase % vs. V₀ | 30.60 | 38.51 |
| inhibition % vs. blank-vehicle | 28.12 | 42.32 |

TABLE 5
Effect of Vehicle III

| Animal N^{o} | V₀ | V₁ | V₂ |
|---|---|---|---|
| 1 | 1.36 | 1.80 | 2.12 |
| 2 | 1.04 | 1.80 | 2.03 |
| 3 | 0.99 | 1.36 | 1.94 |
| 4 | 1.07 | 1.45 | 1.74 |
| 5 | 0.96 | 1.30 | 1.86 |
| 6 | 1.16 | 1.88 | 2.03 |
| 7 | 0.78 | 1.22 | 1.22 |
| 8 | 1.16 | 1.51 | 1.51 |
| 9 | 1.04 | 1.30 | 1.57 |
| 10 | 1.01 | 1.07 | 1.45 |
| Mean ± S.E.M. | 1.06 ± 0.05 | 1.47 ± 0.09 | 1.74 ± 0.09 |

| | | |
|---|---|---|
| increase % vs. V₀ | 28.01 | 39.37 |
| inhibition % vs. blank-vehicle | 34.20 | 41.03 |

TABLE 6
Effect of Vehicle IV

| Animal N^{o} | V₀ | V₁ | V₂ |
|---|---|---|---|
| 1 | 1.07 | 1.74 | 2.26 |
| 2 | 0.93 | 1.36 | 1.30 |
| 3 | 1.01 | 1.39 | 1.88 |
| 4 | 1.07 | 1.07 | 1.59 |
| 5 | 1.13 | 1.94 | 1.74 |
| 6 | 0.93 | 2.03 | 2.17 |
| 7 | 1.07 | 1.74 | 1.62 |
| 8 | 0.87 | 0.93 | 1.54 |
| 9 | 1.25 | 1.51 | 1.74 |
| 10 | 1.16 | 1.36 | 1.30 |
| Mean ± S.E.M. | 1.05 ± 0.04 | 1.51 ± 0.11 | 1.72 ± 0.10 |

| | | |
|---|---|---|
| increase % vs. V₀ | 30.38 | 38.85 |
| inhibition % vs. blank-vehicle | 28.61 | 41.81 |

The group of the comparative results of the percentage of increase of the volume of the paw with regard to V₀ and the percentage of the variation of the edema with regard to the blank-vehicle is shown in the following Table 7.

TABLE 7
Comparative results

| | increase % vs. V₀ | inhibition % vs. blank-vehicle |
|---|---|---|
| Blank-vehicle | 42.56 | — |
| Vehicle I | 37.30 | 12.36 |
| Vehicle II | 30.60 | 28.12 |
| Vehicle III | 28.01 | 34.20 |
| Vehicle IV | 30.38 | 28.61 |

Data obtained show that the composition corresponding to Vehicle I provides a lower inhibition of the inflammation than the inhibition provided by the composition without absorption enhancers (Vehicle IV), due to the absorption of the ibuprofen through the skin. This proves that, unexpectedly, the lactone by its own does not provide an absorption enhancer effect in this type of compositions, but even seems to obstruct said absorption. The composition corresponding to Vehicle II, which comprises oleic acid, provides values of the inhibition of the absorption which are similar to these obtained by the composition corresponding to Vehicle IV, which do not comprise any of the absorption enhancers. Thus, the oleic acid by its own does not provides either a significant effect over the absorption in this type of compositions. However, the percentage of the inhibition of the inflammation with regard to the blank-vehicle obtained when using the composition corresponding to Vehicle III, which comprises a mixture of both enhancers, is higher than expected according to the results obtained with the compositions comprising each one of the enhancers separately. Said superior inhibition of the inflammation shows that the combination of both enhancers has a synergistic effect on the transdermal absorption of the physiologically active agent.

### EXAMPLE 9

### Study of the Anti-Inflammatory Effect of a Composition for Transdermal Administration of the Present Invention which Comprises Oleuropein

Using the method described in Example 7, the anti-inflammatory effect of the four compositions in the form of an oil prepared according to Example 1, which comprised a 0.1%, 0.2%, 0.4%, and 0.8% of oleuropein w/w, respectively, was studied. The blank-vehicle was prepared by the same method as the compositions tested excluding the active ingredient.

The mean±S.E.M. of the volumes of the animal paws was calculated for the three observation periods V₀, V₁ and V₂. The average values of every experimental group were used for the determination of the average increase of the volume of the paws as an effect of the injection of carrageenan during the periods of observation V₁ and V₂ with regard to the initial volume (V₀), and, then, the percentages of the variation of said values with regard to the blank-vehicle group were determined. Finally, the 50% Effective Concentration (EC 50) was calculated through a linear regression optimized by the least squares method. The evaluation of the statistic signification of the variation was performed by the Student's t-test for independent data using a p<0.05 level to establish the difference criteria. The calculation of the EC 50 by the least squares method provides results for the correlation coefficient of 0.93 and 0.89 (for V₁ and V₂, respectively), slightly lower than the unit, which reflects the absence of a concentration-effect correlation at the highest concentrations. However, as estimation, it is obtained that EC 50 of the oleuropein composition in the form of an oil is about 0.7%. The results of these calculations at 60 minutes (V₁) and 180 minutes (V₂) of evolution are shown in Table 8 and 9.

TABLE 8
Anti-inflammatory effect of the oleuropein compositions in the form of
an oil after 60 minutes from the injection of carrageenan (V₁).

| | | inhibition % vs. |
|---|---|---|
| Oleuropein % (w/w) | increase % vs. V₀ | blank-vehicle |
| 0% (blank-vehicle) | 32.08 | — |
| 0.1% | 27.39 | 14.62 |
| 0.2% | 24.75 | 22.86 |
| 0.4% | 18.55 | 42.19 |
| 0.8% | 16.13 | 49.73 |

TABLE 9
Anti-inflammatory effect of the oleuropein compositions in the form of
an oil after 180 minutes from the injection of carrageenan (V₂).

| | | inhibition vs. |
|---|---|---|
| Oleuropein % (w/w) | increase vs. V₀ % | blank-vehicle % |
| 0% (blank-vehicle) | 49.75 | — |
| 0.1% | 43.59 | 12.38 |
| 0.2% | 36.80 | 26.03 |
| 0.4% | 26.80 | 46.13 |
| 0.8% | 25.71 | 48.31 |

According to the obtained results, it can be stated that the anti-inflammatory activity is efficiently developed, i.e. the transdermal absorption is appropriate. Said effect is lineal up to a concentration about 0.4%. The maximum estimated effect of the oily composition of oleuropein is about 50% of inhibition.

### EXAMPLE 10

### Test of Dermal Irritation and Cutaneous Sensitization by Contact Induced by Compositions in the Form of an Oil According to the Present Invention

Following the method described in the Example 1, three compositions in the form of an oil comprising 0.2% w/w of oleuropein (Anti-inflammatory drug I), 1% w/w of ibuprofen (Anti-inflammatory drug II), or 0.2% w/w of oleuropein together with 1% w/w of ibuprofen (Anti-inflammatory drug III), respectively, and a blank-vehicle (composition without active ingredient) were prepared.

A test was performed to evaluate if the application of said compositions on the skin caused some type of irritation or undesired macroscopic changes. The induction of sensitizations that could lead to contact allergies was also evaluated.

28 Wistar male rats, with a weight about 175 g, were used. The animals were acclimatized to the place where the test was performed as described in the Example 7. The test comprised 4 experimental groups, comprising 7 animals each. Blank-vehicle and the compositions designated as Anti-inflammatory drug I, Anti-inflammatory drug II and Anti-inflammatory drug III were applied to the animals.

The test was performed following a modification of the official procedure described in the Official Journal of the European Communities, Dir 961541E, OJL 248, 1996 to study the possible induction of cutaneous sensitizations by the topical application of substances, and the methods to study contact sensitizations of Gregory (Int. Arch. Allergy Immunol. 1993, vol. 101, pp. 72-81), Van der Hoven (J. Pharmacol. Toxicol Methods 1997, vol. 38, pp. 53-57) and Bengt (J. Pharmacol. Exp. Ther. 1999, vol. 88, pp. 1174-1184). The method comprised the application of 100 μl of the substance to be tested over an area of 2×2 cm in the higher third of the depilated back of the rat, slightly massaging the area until total absorption. The substances to be tested were applied every 8 hours for 2 consecutive days (a total of 6 applications), following a blind and randomized pattern. The presence of erythemas, inflammations, desquamations, or other sign or symptom which could indicate a cutaneous aggression or irritation was evaluated 2 hours after the last application.

A week later following the same pattern, the same dose of the reference substances was applied to the lower third of the back of the animals with the objective of induce a contact reaction. Immediately after, a dose of 30 ml/kg of drinking water was administered orally to obtain an optimal hydration of the animal. The presence of undesirable signs or symptoms due to the direct effect of the application of the substance to be tested was evaluated again 24 hours after the application.

None of the tested composition provided noticeable modifications of the dermis nor induced allergic contact reactions.

### EXAMPLE 11

### Study of Preliminary Acute Toxicology by the Composition in the Form of an Oil of the Present Invention

Following the method described in the Example 1, three compositions in the form of an oil comprising 0.2% w/w of oleuropein (Anti-inflammatory drug I), 1% w/w of ibuprofen (Anti-inflammatory drug II), or 0.2% w/w of oleuropein together with 1% w/w of ibuprofen (Anti-inflammatory drug III), respectively, and a blank-vehicle (composition without active ingredient) were prepared.

A test was performed to evaluate if the application of said compositions over the skin provided some type of toxic systemic effect, particularly alterations on the kidney performance, the blood pressure, or the diet.

28 Wistar male rats, with a weight about 175 g, were used. The animals were acclimatized to the place where the test was performed as described in the Example 7. The test comprised 4 experimental groups, comprising 7 animals each. Blank-vehicle and the compositions designated as Anti-inflammatory drug I, Anti-inflammatory drug II and Anti-inflammatory drug III were applied to the animals.

To perform the test, 500 μl of the substance to be tested was applied on an area of 2×2 cm in the higher third of the depilated back of the rat by a slightly massage until total absorption. The substances to be tested were applied every 8 hours for 3 consecutive days (a total of 9 applications), following a blind and randomized pattern, the last application was at 9:30 h of the last day.

None of the tested substances provide noticeable modifications on the cardiac frequency, on the arterial pressure, on the ingestion of food or water, or on the volume of urine excreted. The faecal excretion was neither modified (neither the quantity nor the consistency), although a tendency to a retard of the intestinal emptiness, more noticeable with the anti-inflammatory drug III than with anti-inflammatory drugs I and II, was detected.

What is claimed is:

1. A composition for the transdermal administration of a physiologically active agent, wherein the composition comprises comprising an effective amount of at least one physiologically active agent, at least one vegetal oil, oleic acid and at least one lactone of a saturated or unsaturated fatty acid comprising having from 5 to 22 carbon atoms.

2. The composition according to claim 1, wherein the vegetal oil is present in an amount from about 10% w/w to about 80% w/w, the oleic acid is present in an amount from about 2% w/w to about 50% w/w, and the lactone is present in an amount from about 2% w/w to about 10% w/w.

3. The composition according to any one of the claim 1, wherein the ratio of lactone:oleic acid is from about 0.8:1 to about 1:10.

4. The composition according to any one of the claim 1, wherein the lactone has from 12 to 18 carbon atoms.

5. The composition according to claim 4, wherein the lactone is 15-pentadecanolactone.

6. The composition according to claim 1, wherein the vegetal oil is one or both of a vegetal oil with a content of oleic acid higher than 60% w/w, and a vegetal oil with a content of linolenic acid or another 3-omega fatty acid of at least about 10% w/w.

7. The composition according to claim 6, wherein said vegetal oil is one or both of olive oil and primrose oil.

8. The composition according to claim 1, wherein said physiologically active agent is one or more of oleuropein, an acidic non-steroid anti-inflammatory drug, a polyphenol and a liposoluble vitamin.

9. The composition according to claim 8, wherein the physiologically active agent is oleuropein and the amount of oleuropein is about 0.1% w/w to about 0.4% w/w with regard respect to the total of the ingredients of the composition.

10. The composition according to claim 8, wherein the physiologically active agent is a liposoluble vitamin of one or more of vitamin A, vitamin D, vitamin E and vitamin K.

11. The composition according to claim 8, wherein the physiologically active agent is a non-steroidal anti-inflammatory drug of one or more of indometacin, sulindac, mefenamic acid and derivatives thereof, tolmetin, ketorolac, diclofenac, ibuprofen, naproxen and ketoprofen.

12. The composition according to claim 1, further comprising at least one or more of diethylene glycol monoethyl ether and a polyol having from 2 to 15 carbon atoms and from 2 to 10 hydroxyl groups.

13. The composition according to claim 1, wherein the composition is one or more of an oil, an unguent, an ointment, a cream, an emulsion or a paste.

14. The composition according to claim 1, wherein the composition is an oil comprising from about 20% w/w to about 80% w/w of vegetal oil.

15. The composition according to claim 1, wherein the composition is an unguent comprising from about 10% w/w to about 70% w/w of vegetal oil, and further comprising from about 5.0% w/w to about 25.0% of a melting-point regulator.

16. The composition according to claim 1, wherein the composition is a cream comprising from about 10% w/w to about 50% w/w of vegetal oil, and further comprising from about 1.0% to about 3.0% w/w of at least one emulsifier, and water in an amount sufficient to attain 100% w/w of the composition.
